(19) United States
(12) Patent Application Publication
(10) Pub. No.: US 2021/0361794 A1
(43) Pub. Date: Nov. 25, 2021

(54) Portable Infection Prevention Systems

(71) Applicant: Stephen A. Yencho, Rowlett, TX (US)

(72) Inventor: Stephen A. Yencho, Rowlett, TX (US)

(21) Appl. No.: 17/184,448

(22) Filed: Feb. 24, 2021

Related U.S. Application Data
(60) Provisional application No. 62/980,792, filed on Feb. 24, 2020.

(51) Int. Cl. A61L 2/10; A61L 2/26

(52) U.S. Cl. CPC A61L 2/10; A61L 2/26; A61L 2202/25; A61L 2202/14; A61L 2202/16; A61L 2202/11

(57) ABSTRACT

A handheld disinfection device includes a housing, at least one reflector within the housing, at least one ultraviolet emitter mounted in each reflector, a window that is substantially transparent to ultraviolet light, adjacent to each reflector, through which ultraviolet light emitted by each ultraviolet emitter travels, and at least one position sensor associated with the housing. Another exemplary handheld disinfection device includes a housing, a disinfectant applicator associated with the housing, and at least one position sensor associated with the housing.

This application claims the benefit of priority to U.S. provisional patent application No. 62/980,792, filed Feb. 24, 2020, which is hereby incorporated by reference in its entirety.

### FIELD OF THE INVENTION

This invention relates to devices, systems and methods for disinfecting surface such as in health care facilities.

### BACKGROUND

Nosocomial diseases, also known as Healthcare-Associated Infections (HAIs), are the fourth-leading cause of death in the United States, after heart disease, cancer, and stroke. Over 99,000 patients die each year in the United States of nosocomial diseases, despite the highest annual healthcare spending of any country. (“Multistate Point-Prevalence Survey of Health Care-Associated Infections,” Centers for Disease Control, 2017). Nosocomial diseases are also the most frequent adverse event in healthcare delivery worldwide. (World Health Organization, 2019).

Many pathogens contribute to the nosocomial disease burden. The most common pathogen causing nosocomial disease is Clostridium Difficile (C. Diff). (“Multistate Point-Prevalence Survey of Health Care-Associated Infections,” Centers for Disease Control, 2017). 70.9% of gastrointestinal infections are caused by C. Diff. (“Multistate Point-Prevalence Survey of Health Care-Associated Infections,” Centers for Disease Control, 2017). Other common pathogens causing nosocomial disease include Methicillin-Resistant Staphylococcus Aureus (MRSA), vancomycin-resistant enterococcus (VRE), acinobacter, candida auris, pseudomonas (multiple types), enterobacteriaceae, Enterococcus faecalis, mycobacterium, burkholderia, and Escherichia coli (E. coli). Many of these pathogens are resistant to treatment by antibiotics, making the resultant infections difficult to treat.

Microscopic C. Diff spores and other nosocomial pathogens can be released into ambient air and onto floors and other surfaces, such as through feces of infected patients. C. Diff spores and other nosocomial pathogens can transmit the infectious spores to other patients in the room, as well as to the next patient or patients to occupy the room. Furthermore, they can re-infect a patient in the room. C. Diff spores and other pathogenic organisms can transmit infection to other areas of the healthcare facility or other facility. C. Diff spores can survive up to 5 months on common surfaces such as floors. Each footstep taken by walking on a healthcare facility floor can release up to 100,000 particles into the ambient air, potentially spreading contamination.

C. Diff spores and other cyst-encased pathogens are resistant to disinfection by even the most powerful chemical disinfectants due to the keratin shells which surround the spores and cysts. Advantageously, Ultraviolet-C band (UV-C) light (referred to herein as UV) has been proven effective at inactivating C. Diff and many other disease-causing pathogens responsible for nosocomial diseases. UV light penetrates cyst walls and cell walls to dimerize the DNA of the pathogen, inactivating it to prevent reproduction.

Disinfection systems are currently commercially available which use UV light to inactivate C. Diff and other nosocomial pathogens. Most of these systems act through irradiation of pulsed or constant UV light sources from a central light emitting portable systems to disinfect patient rooms, operating rooms, or other spaces. Due to the harmful effect of UV light on patients and staff, these systems have the common disadvantage that they can only be used in terminal cleaning operations when patients and staff are not present in the room. Other UV disinfection systems use a different approach of continuously radiating small amounts of UV light from permanent ceiling-mounted fixtures while simultaneously radiating visible light to the room from the same fixture.

Since an infected patient can generate C. Diff spores and other pathogens while occupying a patient room or other area of a healthcare facility, existing systems cannot prevent re-infection of the patient nor prevent infection of other room occupants. Furthermore, C. Diff spores and other nosocomial disease organisms generated by the patient in the room may escape from the room to infect other areas.

Most existing portable UV room disinfection systems operate from a central location in each room, causing the radiated UV light to travel distances of 3 to 4 meters or more for all room surfaces to be illuminated by UV light. UV light is strongly absorbed by oxygen molecules in the air, causing UV light intensity to substantially decrease with distance from the light source. Furthermore, the physics of light irradiation cause light intensity to decrease by the square of the distance from the light source. Portions of a patient room may not receive direct UV irradiation during disinfection treatment from existing portable UV systems, and must rely on less intense reflected UV light for disinfection. Many typical healthcare facility surfaces such as painted walls and tiled floors are poor reflectors of UV light. For these and other reasons, recent studies of central room UV irradiation systems have demonstrated that over 30% of nosocomial pathogens can survive in a patient room even after treatment by existing UV disinfection systems. This level of disinfection performance is far too low to ensure patient safety and prevent nosocomial disease transmission.

All of these factors illustrate the critical need for a new, effective UV disinfection system which can both inactivate C. Diff and other nosocomial pathogens to a high degree of efficacy and be safely used while patient(s) and hospital staff occupy the hospital room or operating room. Advantageously, the system of the present invention provides a high degree of disinfection while enabling use while the room is occupied. In addition, the system of the present invention may be used for other applications such as hotel room cleaning, hotel bed cleaning, health club disinfection, school bathroom cleaning, and other applications.

Few accurate methods exist for rapidly determining disinfection efficacy following disinfection procedures. Swabbing of potentially contaminated surfaces followed by 24 hour incubation of the swabbed samples is one of the most effective techniques to determine the presence of C. Diff spores. Due to the effort required to obtain sufficient numbers of samples for each disinfected surface or room, and the time and cost required to incubate these samples, swabbing and similar techniques are not commonly employed to verify proper surface disinfection. Therefore, hospital housekeeping staffs usually have no accurate information as to the true state of disinfection of a hospital room, even after full cleaning and room-level UV light disinfection treatments.

### SUMMARY OF THE INVENTION

According to some embodiments, a handheld UV disinfection device may produce up to 1,000,000 times more UV light intensity at the disinfection surface than existing whole room UV machines or robots, due to the intensity of the UV light sources and their proximity to the surface. Advantageously, the disinfection device may have minimal UV edge leakage to allow it to be used while patient and/or staff are present in the room, without creating a hazard to them. In a battery-powered configuration, there are no power cords to cause trip hazards. The disinfection device may be used in single-pass or multiple-pass surface disinfection treatments. Optionally, one or more chemical disinfectants, such as but not limited to bleach and/or glutaraldehyde, may be dispensed from a reservoir associated with the disinfection device. Alternately, or instead, disinfection treatment may be preceded by wiping down the surface with a chemical disinfectant.

According to some embodiments, the disinfection device is used to disinfect surfaces in a hospital room or other area, and a disinfection system incorporates multi-dimensional spatial tracking that acquires data to simulate disinfection performance. The simulation is performed after disinfection, and generates useful information regarding the state of disinfection of the hospital room or other area that was disinfected.

According to some embodiments, the disinfection simulation results are displayed as a visual overlay on the space to be disinfected. The spatial dimensions tracked by the system include at least one set of positions in three dimensions of space: X, Y, Z, as well as the angular orientation around each of the three X, Y, Z axes, and the dimension of time for each position. The spatial tracking information in combination with the computed pathogen kill rate of the disinfection device is preferably used as input to a disinfection simulation model. Instantaneous spatial position of the disinfection device, UV lamp power levels, distance of the disinfection device from the surface, and other critical variables may be stored and used as inputs for a simulation model to calculate disinfection performance point by point over the surfaces that were disinfected. The UV disinfection performance is computed by combining the computed instantaneous velocity data and disinfection device orientation at each point on the surface with the known UV luminous power over the illuminated area at the disinfection surface. The relationship of UV fluence to the log kill rate of a specific pathogen is then applied at each surface point to obtain the pathogen log kill rate at each point on the surface. The log kill rate at each point may be converted to a specific color or grayscale shade for disinfection display purposes. The entire array of log kill rates at each surface point may be overlaid over the surface area for visualization. Alternately, the disinfection simulation image may be displayed on a display on the disinfection device itself, on a computer screen, or tablet computer, or other device.

The disinfection simulation model may be displayed on a mixed reality/augmented reality (collectively, “AR”) headset as an overlay to the disinfected area. Alternately, they may be displayed on a screen on the disinfection device. Alternately, they may be displayed on a display on the disinfection device itself, a computer display, handheld tablet, smart phone, projector, or other device. The dataset and/or the results of the simulation model may be stored in a database or memory for later use. In this way, a user may visualize the calculated results of the disinfection that was performed.

The disinfection simulation model may be calibrated by using the disinfection system to disinfect a pre-contaminated surface and obtaining samples taken from the disinfected surface to determine the pathogen kill rate at several points on the surface. By comparing these disinfection results with the captured spatial tracking data and computing velocity of the system over the surface and summing the cumulative UV exposure at each point on the surface, an accurate model of the pathogen kill rate may be created. The disinfection calibration data obtained by swabbing and incubating samples from surfaces after disinfection by the disinfection device may be used to enhance the disinfection simulation model, and/or to change the disinfection requirements for specific hard-to-disinfect areas. Calibration may also include measuring actual UV output at each point under disinfection device and summing that UV fluence info to each point of the tracked path taken by the device over the surface.

The spatial distribution of the pathogen kill rate can be overlaid over the physical space for visualization, either on a computer monitor or handheld tablet or with a mixed reality headset for direct visualization of the specific degree of disinfection in room or other location. The data may be compared to previous disinfection performance data sets for the same space to generate statistical disinfection process control information for the facility. The historical disinfection performance data may be used as inputs for machine learning algorithms to obtain feedback on improving disinfection performance. The disinfection results from each cleaning may be used to optimize disinfection protocols using machine learning algorithms.

Disinfection data from the system can be combined with historical disinfection data to obtain statistical process control information. Due to the lack of accurate information, statistical process control systems are not often used to take information from disinfection operations and verify statistical disinfection performance. Disinfection data may be correlated with patient data to predict probabilities of specific infections. For example, historical disinfection data for a patient unit may be compared to create R Bar Charts to maintain the patient unit in accordance with statistical process control procedures.

The disinfection performance of other disinfection devices, such as existing whole room UV robots or devices, may be computed based on a disinfection simulation model with knowledge of the position and intensity of the UV source in the room and knowledge of the UV absorption rate of the ambient air and each surface. The pathogen kill rate may be computed and displayed for each point in the room by taking into account wall and ceiling UV ray reflections and UV absorbance of atmosphere in room along the light path. Similarly, sponges, mops, and other cleaning and disinfection products may be equipped with tracking sensors to acquire data for computing and displaying disinfection or cleaning results.

According to some embodiments, three-dimensional spatial tracking data is acquired during the disinfection process and the corresponding disinfection performance is computed over the disinfected space using the spatial tracking data. This method may be useful in other applications. For example, it may be used with appropriate sensors in biological warfare applications and other applications of infectious disease control. When used in combination with the appropriate type of sensor(s), the method can be used in chemical decontamination and nuclear decontamination applications, as well as in surface coating and surface treatment applications or airplane deicing applications. The method can also be used to visualize liquid plumes, such as for chemical spills into bodies of water.

### BRIEF DESCRIPTION OF THE DRAWINGS

FIG. 1 is a perspective view of an exemplary handheld surface disinfection device.

FIG. 2 is a bottom perspective view of the exemplary handheld surface disinfection device of FIG. 1.

FIG. 3 is a cross-section bottom perspective view of the exemplary handheld surface disinfection device of FIG. 1.

FIG. 4 is a cross-section bottom perspective view of the exemplary handheld surface disinfection device of FIG. 1, with one of the UV lamps removed.

FIG. 5 is a bottom perspective view of another exemplary handheld surface disinfection device.

FIG. 6 is a bottom perspective view of a floor disinfection device.

FIG. 7 is perspective view of an autonomous surface disinfection device.

FIG. 8 is an exemplary electronics block diagram for any of the embodiments of disinfection devices above.

FIG. 9 is a perspective view of a sponge or wipe with integrated sensors.

FIG. 10 is a perspective view of a mop with integrated sensors.

FIG. 11 is a flowchart describing a spatial tracking and data logging algorithm

FIG. 12 is a diagram of an exemplary set of tracked points from a disinfecting device with assigned grid points.

FIG. 13 is a transducer communication diagram.

FIG. 14 is a floor plan of a disinfected area, including transducers.

FIG. 15 is a floor plan of a disinfected area, including transducers and computed deficient disinfection areas

FIG. 16 is a perspective view of a head-mounted visual display on a user.

FIG. 17 is a visual display with augmented reality overlay of spatial disinfection data.

FIG. 18 is a perspective view of another exemplary handheld surface disinfection device in a first, stowed position.

FIG. 19 is a perspective view of the exemplary handheld surface disinfection device of FIG. 18 in a second, opened position.

FIG. 20 is a cutaway view of the exemplary handheld surface disinfection device of FIG. 18 in a second, opened position.

FIG. 21 is a perspective view of another exemplary handheld surface disinfection device that includes a sponge.

FIG. 22 is a cutaway view of the exemplary handheld surface disinfection device of FIG. 21.

The use of the same reference symbols in different figures indicates similar or identical items.

### DETAILED DESCRIPTION OF THE INVENTION

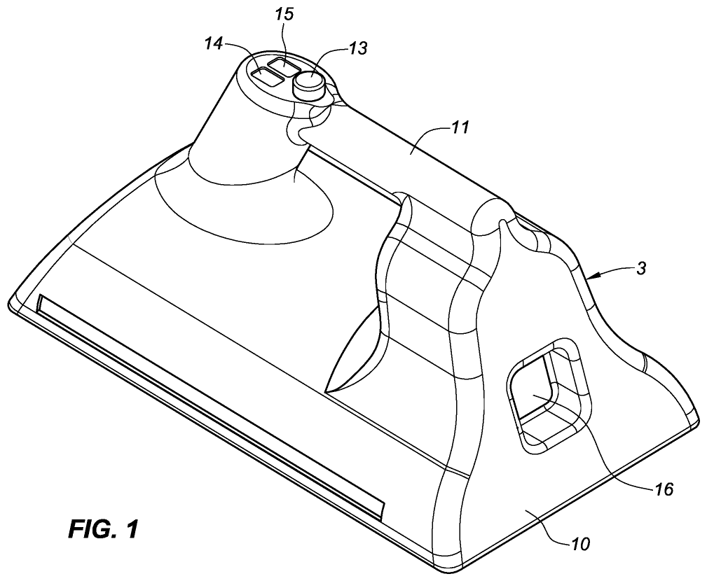

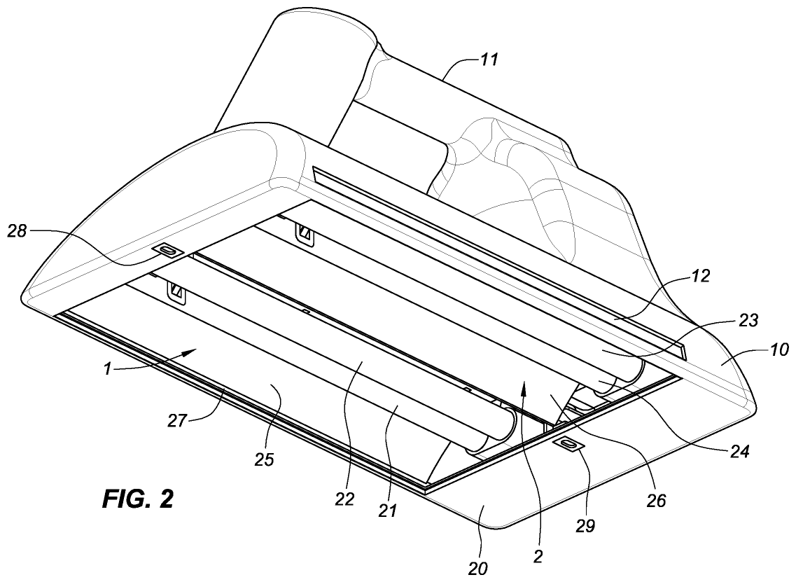

An exemplary handheld UV disinfection device 10 is shown in FIG. 1. The disinfection device 10 may include a housing 3. The housing 3 may include a handle 11 by which a user holds the disinfection device 10; the handle 11 may form part of the housing. An on/off switch such as a momentary pushbutton switch 13 activates UV lamp tubes 21, 22, 23, and 24, as shown in FIG. 2. Display 14 with soft key(s) or other pushbutton(s) 15 may enable the user to make selections from a set of displayed menu options to calibrate and perform other functions. Display 14 is preferably able to provide real-time feedback to the user to show whether the surface speed is below the maximum speed for proper disinfection and may also be used to direct the operator in real time to adjacent areas needing further disinfection. Display 14 also, or instead, may be able to display battery level, where the disinfection device 10 is battery-powered. Handheld disinfection device 10 may be equipped with a recharging port 16, or alternately may use rechargeable batteries which plug in to handheld device 10. According to other embodiments, the disinfection device 10 may be configured with a cord and plug that may be plugged into a standard electrical outlet as a power source.

The disinfection device 10 may include one or more UV lamps. A first UV lamp 1 containing lamp tubes 21 and 22 may be mounted in a first semi-parabolic reflector 25. A second UV lamp 2 containing lamp tubes 23 and 24 may be located in a second semi-parabolic reflector 26. Alternately, a single UV lamp may be used, or more than two UV lamps may be used. The UV lamp tubes 21, 22 and 23, 24 are preferably located along the axis of the respective parabolic reflectors 25, 26. The parabolic reflectors 25, 26 are preferably made of aluminum. The aluminum reflectors 25, 26 may be coated or anodized as required. Alternately, reflectors 25, 26 may be made of stainless steel or another suitable material, and/or may have a cross-section other than a parabolic one. Rows of UV-emitting LEDs may be used instead of, or in addition to, the UV lamp tubes 21, 22 and 23, 24. As one example, UV lamps 1 and 2 may each consume 24 watts of electrical power and produce 7.1 watts of UV-C power. Alternately, a different input power level may be applied and/or a different wattage of output power may result.

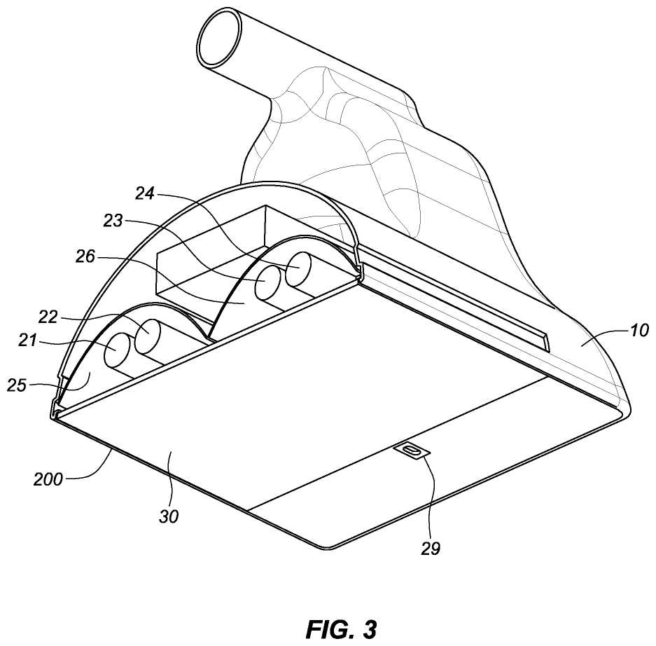

Referring also to FIG. 3, the handheld disinfection device 10 may include a UV-transparent window 30. Window 30 is preferably made of fused quartz, fluorinated ethylene propylene (FEP) film, colorless polyimide, or other suitable UV-C-transparent material. The window 30 protects the lamps 1, 2 from inadvertently coming into contact with irregular surface to be sanitized, and therefore protects the lamps from damage. The window 30 may be positioned apart from the surface to be disinfected a distance of less than 2 mm. To do so, the window 30 may be supported by an edge 200 of the handheld disinfection device 10, which holds the window 30 at a distance of 2 mm or less from the bottom surface of that edge 200. In other embodiments, three of more UV-C-transparent contact points may extend downward from the window 30, where those contact points are 2 mm or less in length. Lamp tubes 21, 22, 23, and 24 are preferably enclosed in a shatterproof sheath or alternately have a shatterproof costing such as FEP, polyvinylidene fluoride (PVDF), polytetrafluoroethylene (PTFE), or colorless polyimide to keep them from shattering if the handheld disinfection device 10 is dropped. Alternately, another suitable material is used. Preferably, the handheld disinfection device 10 has properly positioned shock absorbing bumpers to absorb impact if dropped. According to some embodiments, the window 30 may be positioned further from the surface to be disinfected than 2 mm, or may be located substantially flush with the surface to be disinfected.

Referring also to FIG. 2, optical position sensors 28 and 29 in FIG. 2 each provide two-axis location data for disinfection device 10 on a surface. Optical position sensors 28 and 29 may be of various designs. According to some embodiments, optical position sensors 28 and 29 include a laser source. Alternately, an LED light source may be used. Optical position sensors 28 and 29 are may illuminate the surface through a lens, and each include one or more phototransistors or other sensors configured to receive the reflected illumination light through a lens. Alternately, the illumination source and the sensors may be separate components and/or separately mounted on the handheld disinfection device 10. Advantageously, the optical position sensors 28 and 29 are unaffected by the UV light emitted by the disinfection device. Optical sensors 28 and 29 may be similar in design to conventional computer mouse optical sensors. They preferably are configured to sense high speed motion of at least 1 m per second (40 inches per second), and high accelerations of up to 25 G. Preferably, they are able to measure at least 15 or more counts per mm (400 counts per inch) of motion. Alternately, another resolution may be chosen. Optical sensors 28 and 29 are preferably have internal fault detection circuitry for eye safety compliance. Optical sensors 28 and 29 are preferably located equidistant from the center of the UV illuminated window 30 on the bottom of the disinfection device 10.

Optical position sensors 28 and 29 advantageously are able to track even smooth and uniform surfaces such as stainless steel which are prevalent in health care settings. The optical position sensors may be registered at a home location on a surface to be disinfected. Alternately, optical position sensors 28 and 29 may self-calibrate as they pass over pre-arranged optical patterns on the floor or other surface to eliminate the need for homing. The optical patterns on the surface may consist of fine lines so as to be nearly invisible to the human eye.

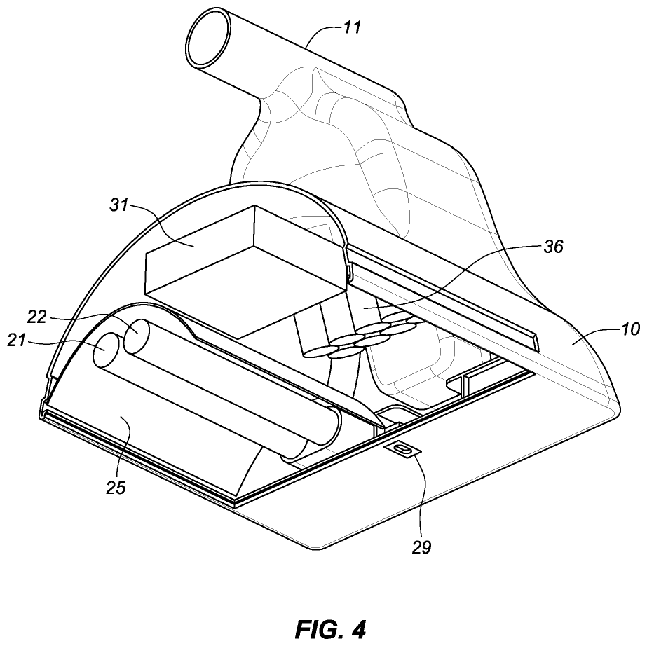

Referring also to FIG. 4, one or more batteries 36 and an electronics module 31 are located in the handheld disinfection device 10 as shown in FIG. 4. Preferably, lithium-ion batteries, such as an array of 18650 batteries, may be used to power handheld disinfection device 10. Alternately, lithium phosphate batteries or other suitable high capacity, lightweight rechargeable batteries may be used. Electronics module 31 is preferably thermally isolated from the UV lamps and other heat sources in the disinfecting device 10. Electronics package 31 may employ a fan for cooling, or may be passively cooled.

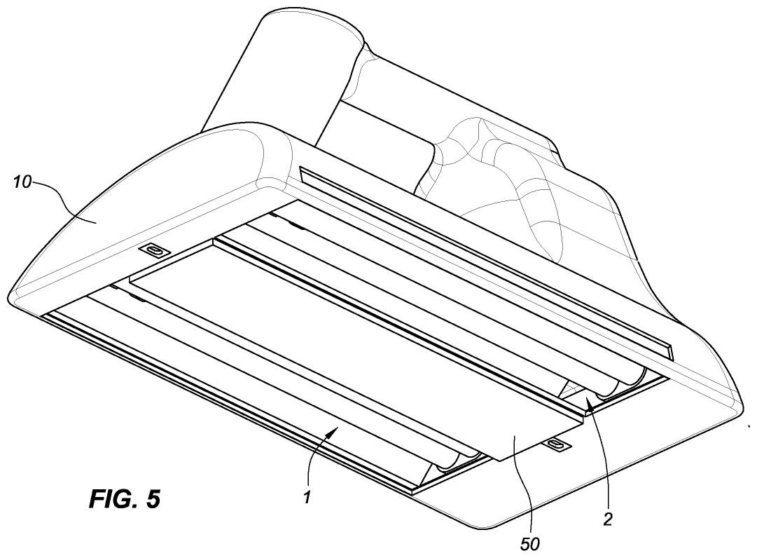

According to some embodiments, referring also to FIG. 5, the handheld disinfecting device 10 may be equipped with a central liquid- or vapor-dispensing disinfectant applicator 50. The disinfectant applicator 50 may be positioned laterally between lamps 1, 2, such that the lamps 1, 2 are spaced apart laterally. The disinfectant applicator 50 may be positioned on the window 30 if used, or on a surface or structure positioned between the lamps 1, 2 if the window 30 is not used. The disinfectant applicator 50 may be active, meaning that the disinfectant dispenser may include a reservoir of disinfectant liquid or gas that is dispensed manually by pressing a button or actuating another control. Alternately, disinfectant may be dispensed automatically as needed or as determined from a disinfection simulation. According to other embodiments, the disinfectant applicator 50 may be passive, such that a reservoir of disinfectant is not provided in the handheld disinfecting device 10, and the integrated disinfectant applicator 50 wipes disinfectant from the surface, or is soaked in disinfectant prior to use. Examples of passive disinfectant dispensers include wipes and sponges. In such embodiments, the disinfectant applicator 50, or the surface to be disinfected, may be sprayed or otherwise may receive disinfectant periodically. The use of liquid disinfectant prior to, during, or after the application of UV light to the surface to be disinfected may provide additional efficacy of disinfection of that surface.

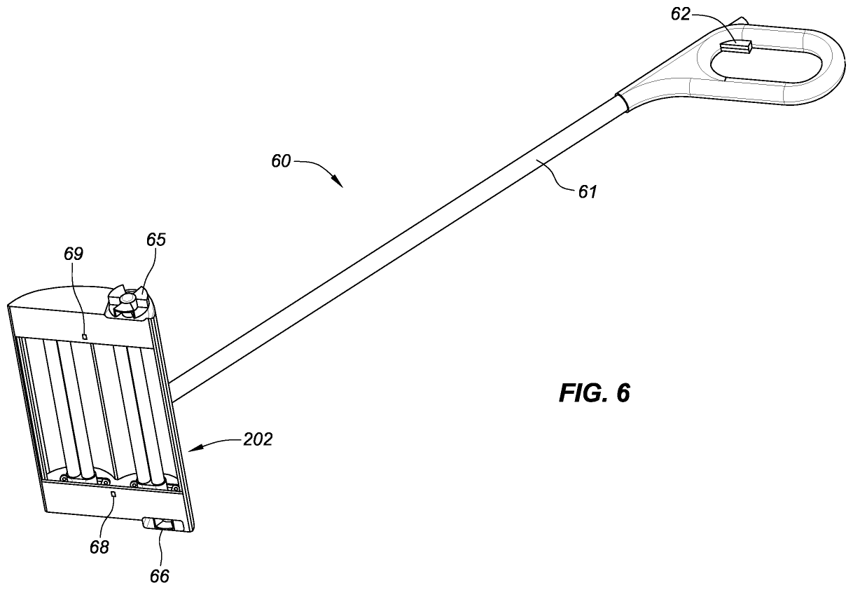

Referring to FIG. 6, an exemplary floor disinfection device 60 is shown. The floor disinfection device 60 may include a handle 61 with on/off trigger 62 and operator display and softkeys (not shown). The handle 61 attaches to a disinfection unit 202, which is substantially the same as the embodiments of the handheld disinfection device 10 described above, with the primary differences described here. Wheels 65 and 66 enable the disinfection unit 202 to roll easily across the floor. Wheels 65 and 66 preferably surround a central UV lamp or UV LEDs. Alternately, wheels 65 and 66 are illuminated by a UV lamps or UV LEDs. Wheels 65 and 66 are preferably UV-transparent to enable inactivation of pathogens on their surfaces and prevent the wheels from spreading active pathogens across the floor. Wheels 65 and 66 may be made of suitable UV-transparent material such as fused quartz, FEP, colorless polyimide, or other polymer. Alternately, wheels 65 and 66 may have UV LEDs arrayed around their circumference. Optical position sensors 68 and 69 each provide two-axis location data for the disinfection unit 202 on a surface. An array of 3-D accelerometers and 3-D gyroscopes may be included in the disinfection unit 202 to provide further positioning information. An ultra-wideband (UWB) transponder may be included in the disinfection unit 202 in combination with other anchor UWB transponders, as described in greater detail below. A wireless radio transceiver may be included in the disinfection unit 202 to enable communication over Wi-Fi or Bluetooth to transmit information between the disinfection unit 202 and fixed transceivers. The disinfection unit 202 optionally can dispense and wipe liquid or vapor disinfectant.

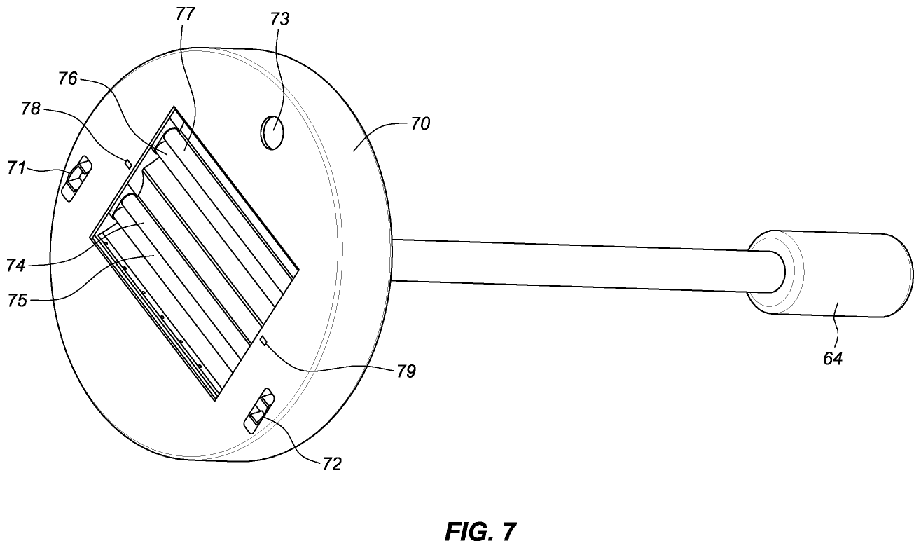

Referring to FIG. 7, an exemplary embodiment of an autonomous disinfecting device 70 is shown. The autonomous disinfecting device 70 may be substantially the same as the embodiments of the handheld disinfection device 10 described above, with the primary differences described here. The autonomous disinfecting device 70 may include motor driven wheels 71, 72, and a caster wheel 73, and may be able to move across surfaces such as floors without a human operator. Optional encoders on wheels 71 and 72 may be used to track location and speed. UV lamp tubes 74, 75, 76 and 77 in parabolic reflectors emit UV light, as in the previous embodiments. A UV-transparent window may cover the lamp area, as described above with regard to the window 30 of the handheld disinfection unit. Optical position sensors 78, 79 each provide two-axis location data for disinfection device 10 on a surface. Preferably, a safety beacon 64 provides a flashing light to indicate the presence of the disinfection device. An optional display displays the status of the unit. As described above with regard to FIG. 6, drive wheels 71, 72 and caster wheel 73 preferably contain a UV lamp and/or are UV-transparent to prevent them from spreading active pathogens along the floor. The autonomous disinfecting device 70 preferably employs real-time tracking. The disinfection device optionally can dispense and wipe liquid or vapor disinfectant, as described above with regard to FIG. 5.

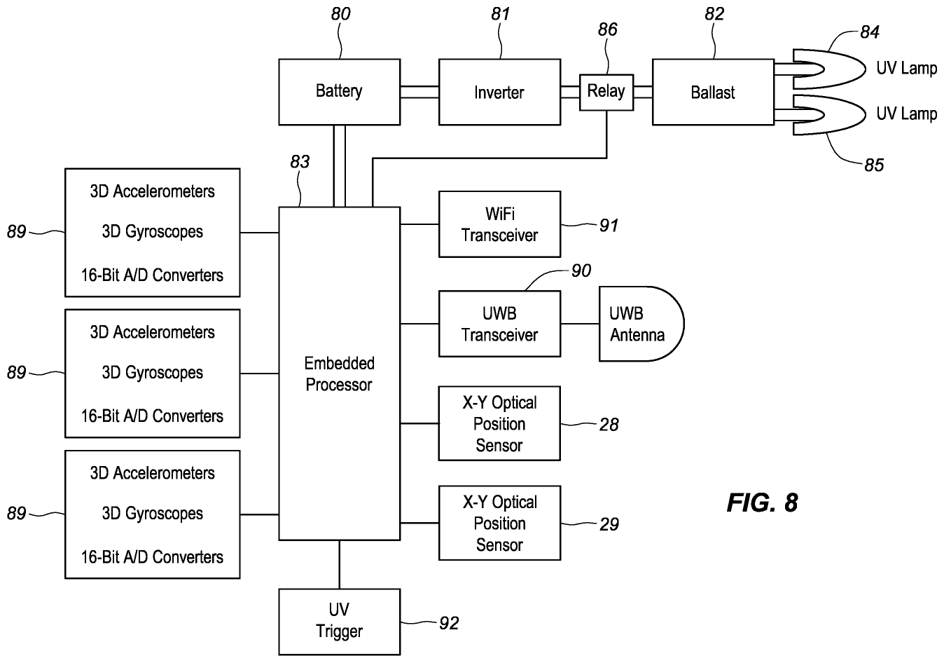

Referring to FIG. 8, an exemplary electronics block diagram for the handheld disinfection device 10 or floor disinfection device 60 is shown. Robotic disinfection device 70 preferably uses a similar electronics module with additional functionality including power electronics to drive motors for wheels 71 and 72. A rechargeable battery 80 provides power for inverter 81 and electronics module 83. Ballast 82 drives UV lamps 84 and 85. A relay 86 enables lamps 84 and 85 to be turned off. Electronics module 83 preferably contains an embedded microcomputer such as an ARM or equivalent and is interfaced to a set of sensors as shown in FIG. 8. Optical sensors 28, 29 preferably have self-contained LEDs and phototransistors or vertical cavity surface effect lasers to accurately capture x and y movements along the surface. An array of 3-D accelerometers and 3-D gyroscopes 89 provides further positioning information. An ultra-wideband (UWB) transponder 90 may be used in combination with other anchor UWB transponders as described below. A wireless radio transceiver 91 enables communication over Wi-Fi or Bluetooth to transmit information back and forth to fixed transceivers, including data from optical sensors 28 and 29, and accelerometers and gyroscopes 89, as needed.

Improved accuracy may be obtained by using an array of several identical 3D accelerometers and 3D gyroscopes 89, and by averaging the noisy data. Advantageously, 3D accelerometers and 3D gyroscopes 89 are available on surface mount integrated circuits which are very compact and have low power requirements, allowing mounting even on a sponge or other small cleaning objects; can get good spatial location accuracy for disinfection model. Both accelerometers and gyroscopes 89 are preferably thermally compensated to improve accuracy. Preferably, the accelerometers and gyroscopes 89 are shock mounted for impact protection. UV lamp ON/OFF trigger 92 is preferably monitored with spatial position to know for which spatial locations the UV lamp was on and off and as an indicator that the disinfection device is being moved to a new surface. Optionally, electrical current to the lamp(s) can be tracked by Hall Effect sensors to provide UV fluence data.

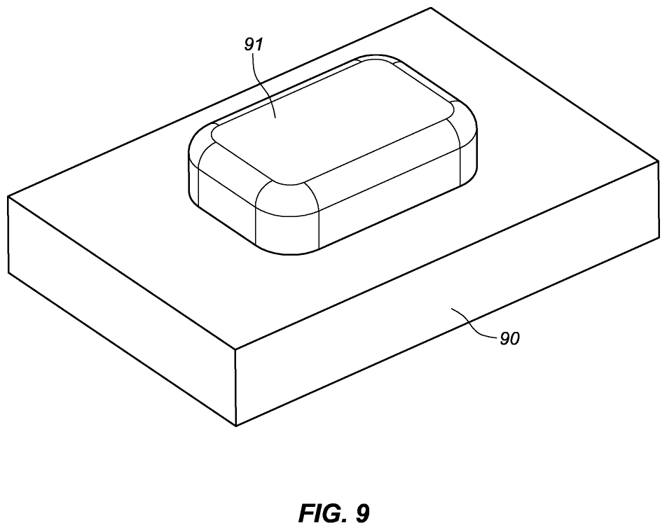

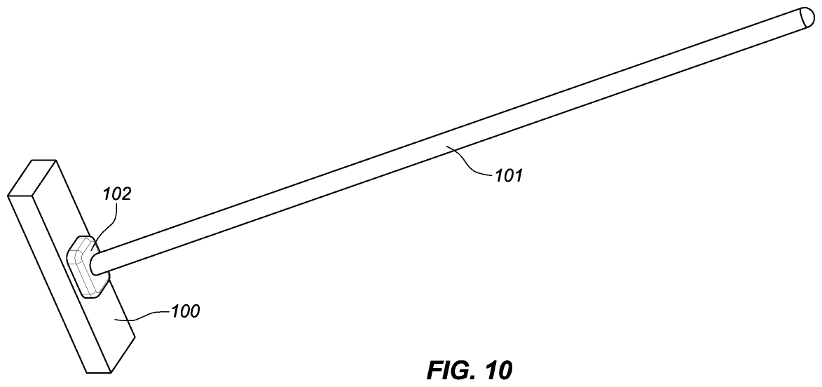

In another aspect of the invention, liquid disinfectant dispensing and/or wiping products such as sponges, mops, and wipes may be similarly equipped with tracking sensors and tracked to observe effectiveness of the disinfection process. Referring also to FIG. 9, an exemplary sponge 90 or wipe with integrated 3D accelerometers and 3D gyroscopes and a wireless transceiver may be attached to a waterproof housing 93. A key indicator of liquid disinfectant performance is the amount of time that the disinfectant remains on the surface. Therefore, the position data and the elapsed time tracked between applying the disinfectant to the surface with a sponge and wiping the same location with a wipe can be used as inputs to a disinfection simulation model resulting in a pathogen log kill display of disinfection performance across the surface. The position data associated with the wiping of the sponge 90 or wipe on a surface, and the time that the sponge 90 or wipe occupied any given point on a surface, may be determined in any suitable manner. For example, an optical sensor 28, 29, as described above, may use a laser diode to generate a laser that reflects from the surface being cleaned. The reflection from the laser is raw two-dimensional position data, which may be transmitted to the electronics module 83. The position data may include the time at which the optical sensor 28, 29 occupied a particular position. The position data and time data may be used to determine how much time during the cleaning process that the sponge 90 or wipe was located at a particular point, or a particular area, of the surface to be cleaned. Referring also to FIG. 10, a mop 100 with handle 101 and integrated 3D accelerometers and 3D gyroscopes and a wireless transceiver may be located in a waterproof housing 102. The mop 100 functions similarly to the sponge 90 or wipe described above.

A variety of options exist for tracking and communicating with the disinfection devices. Tracking may be internal based on optical surface sensing, accelerometers, gyroscopes, magnetometers, or other sensors, or external, based on ultra-wideband (UWB) transponders or other wireless tracking techniques. Sensors located in the disinfection device are preferably used to track the device. The disinfection device is preferably physically registered to a specific location on the surface to enhance accuracy.

The surface registration procedure may require registering the disinfection device at the same location on the surface with two orientations of the disinfecting device (ideally perpendicular to each other) to eliminate the accelerometer bias caused by the skewed magnetic field of the earth relative to a horizontal surface which may be 60 to 70 degrees from vertical in the US. A permanently positioned home position label, preferably located on the floor of each room, preferably is a wireless UHF RFID tag with angle sensing capability. Such a tag may be as described in Xiaozheng Lai, et al, A Novel Displacement and Tilt Method Using Passive UHF RFID Technology, Sensors 18, 1644, 21 May 2018, and may be referred to as a UHF-Theta tag. The tag position and orientation may be sensed by a transceiver on disinfection device 10. The tag is preferably oriented in the X- and Y-axes with the room in accordance with the expected disinfection simulation. Alternately, the home tag may be an adhesive label with a home position and X-axis and Y-axis lines that correspond to a home point and X-axis and Y-axis orientation lines on the disinfection device. Preferably, a UHF-Theta tag is placed each piece of furniture or equipment, since furniture and equipment is often moved around the healthcare facility. Alternately, the home position label may include orientation information provided by an embedded magnet which is detectable by the disinfection device and allows determination of equipment orientation. An embedded magnet may be used in floor homing tags as well for the same purpose. A momentary calibration pushbutton is preferably depressed by the operator when the disinfection device is properly positioned for calibration. Simultaneous mapping of the environment with spatial location determination using 3D accelerometers and 3D gyroscopes and 3D visual and 3D infrared camera-based navigation may be used to acquire and register initial spatial locations, therefore eliminating the need to home to a tag or label on each surface.

Because surface reflectivity can affect the pathogen kill rate, it is important to include its effect in the disinfection algorithm described below. Selection of the proper surface type improves the disinfection simulation performance by more accurately characterizing the UV reflection rate to compute accurate pathogen disinfection rates. Referring also to FIG. 2, the surface type may be selected from a set of menu options on display 14. Alternately, the surface type may be detected by monitoring the reflected power levels at the optical position sensors 28 and 29. Alternately or in addition, metallic surfaces may be detected by using Hall effect sensors located near the surface. Alternately, the surface type may be detected by using a camera.

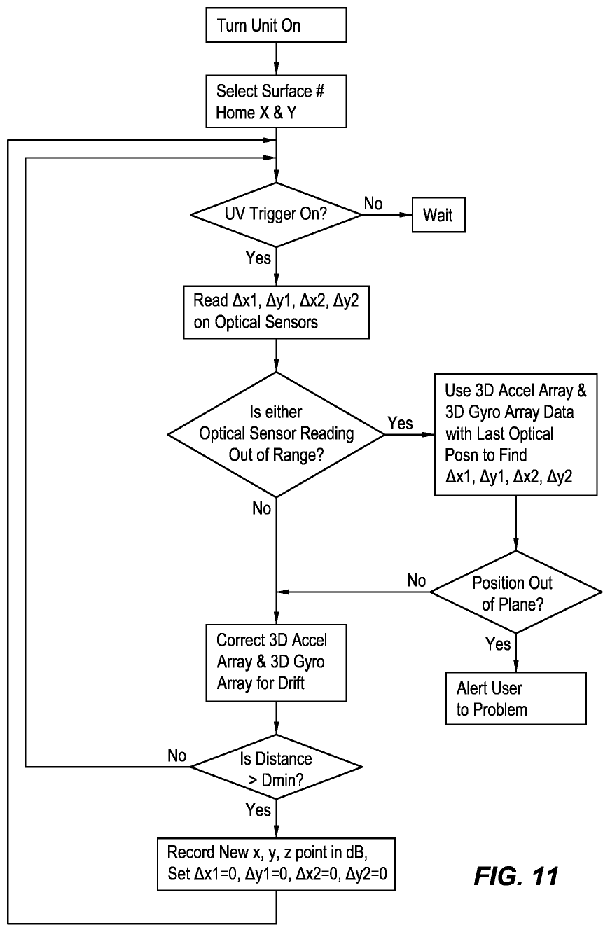

FIG. 11 shows a flowchart of a spatial tracking and data logging algorithm. This simple exemplary algorithm is based on the following assumptions:

1. The surfaces to be disinfected are planar.
2. Each planar surface has a marked homing location with marked X and Y homing orientations.
3. The operator depresses the UV trigger when the disinfection device is ready to home and keeps the UV trigger depressed until the entire surface is disinfected. The UV trigger is released to turn UV lamps 82 off by action of relay 86 at the completion of each surface disinfection.

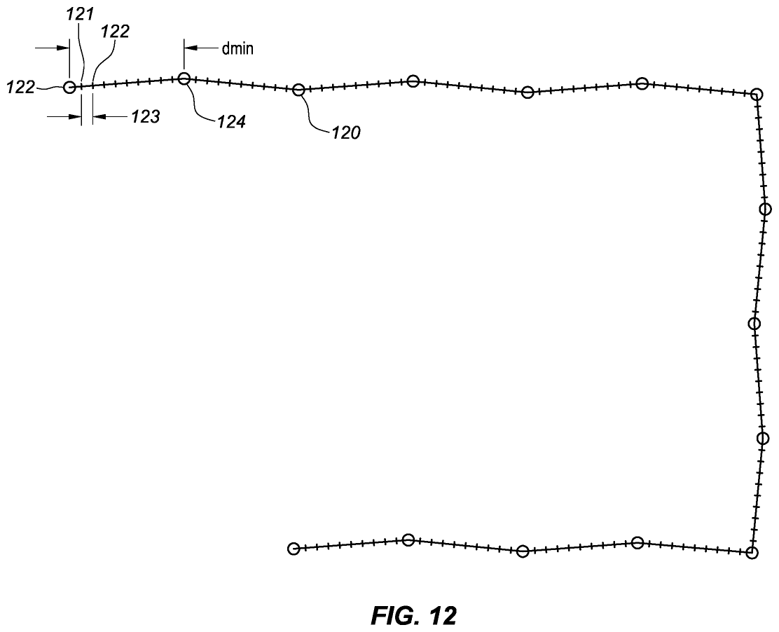

As shown in FIG. 11, the spatial location is tracked using optical position sensors 28 and 29 and by continuously monitoring both the thermally compensated 3D accelerometers and the thermally compensated 3D gyroscope arrays 89 to track the position and orientation of the disinfection device from the home position. In this way, the algorithm in FIG. 11 compensates for gyroscope drift. Incremental displacements Δ x1 and Δ y1 are obtained by polling optical sensor 1. Incremental displacements Δ x2 and Δ y2 are obtained by polling optical sensor 2. These incremental displacements can correspond, for example, to a moving the centroid of the UV window from point 121 to point 122, for a total distance of 123 in FIG. 12. When the distance moved by the centroid of the UV window from starting grid point 122 is equal to or greater than dₘᵢₙ, a new grid point 124 is recorded in the disinfection path array.

In the event of a discontinuity or a fault condition from one or more of the optical sensors 28 and 29, the 3D accelerometer and 3D gyroscope arrays 89 are used to obtain position information for the current point in space. The current 3D position is computed by digitally double integrating the acceleration data from the 3 axes of the accelerometers to get 3 axis position displacement information. The 3D orientation data is obtained by digitally integrating the rate of rotation data from the 3D gyroscope array to get angular displacement information for each of the three axes. The algorithm regularly corrects for potential drift of the gyroscopes when relying on the optical sensors. In this way, the path tracking array is constructed in memory, as shown in the flow chart in FIG. 11.

The accelerometer array data and the gyroscope array data are preferably averaged or otherwise conditioned to compensate for noise. Preferably, multiple sets of 3-D accelerometers and 3-D gyroscopes 89 are used to improve accuracy. As many as 6 sets of 3-D accelerometers and 3-D gyroscopes 89 may be used simultaneously to improve accuracy. Data may be transferred to another computer using Wi-Fi transceiver 90. Alternately, a Bluetooth transceiver or other means is used.

Instead of relying primarily on the 3D accelerometers and 3D gyroscopes, the disinfection device may be tracked by radio frequency transponders such as ultra-wideband (UWB) transceivers. In this embodiment, the disinfection device contains a UWB transponder which communicates with a set of small, preferably wireless UWB transponders mounted in known locations in each room. The 3-D spatial location of the disinfection device may be measured using the UWB transceivers by several methods, including Time-Difference-Of-Arrival (TDOA), Two-Way Ranging (TWR), Time Of Flight (TOF) or other methods using the array of UWB wireless transducers.

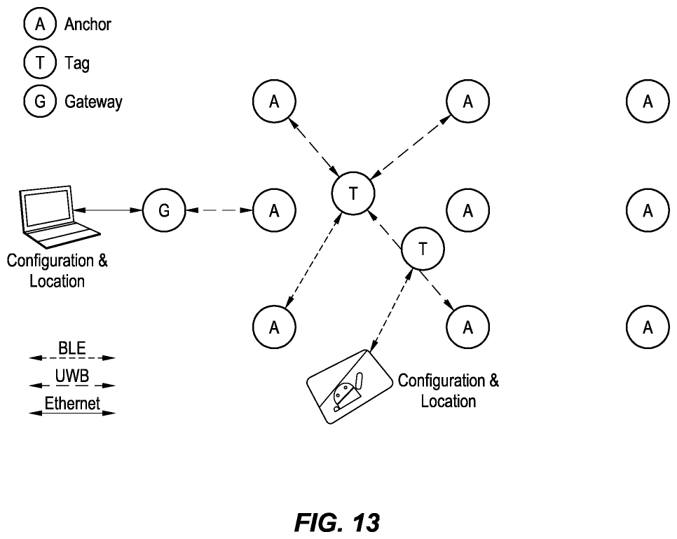

Preferably, at least four UWB transponders are placed in each room to minimize interference effects of signals passing through walls. The transducers communicate as shown in FIG. 13. Preferably, one or more UWB transducers are also mounted to each bed, each piece of furniture, tables, etc. to monitor their location in the room and to monitor the position of the disinfection device(s) relative to them. The transponders may be wireless and battery-powered or wired to building electrical system and wired with Ethernet or other communication cabling. The UWB transponders operate in the frequency range from 2 GHz to 7 GHz. The accuracy of tracking the disinfection device with UWB transponders can be down to centimeters or even millimeters.

3-D Position and status information and accelerometer and gyroscope data from disinfection device 10 may be transferred wirelessly to a specific UWB transponder in the room, which is configured to function as a gateway for transmission of data over the Internet. Using this method, computed spatial tracking array data may be transferred over the UWB transceivers.

Magnetometers may also enable 3-axis tracking using the earth's magnetic field or an artificial magnetic field as a reference. However, the magnetic field received by the sensors may be distorted by interference from steel building structure or other large ferromagnetic objects. This effect can be compensated since the building structure is stationary. In the preferred embodiment, magnetometers are not required.

With an improved sensing algorithm, the need to identify the surface to be disinfected and the need to home the disinfecting device may be eliminated by more accurate 3D position tracking. For example, one or more cameras may be mounted on the disinfection device to track the spatial position of the device during use and to predict disinfection performance. Machine learning systems can use video images to guide the operator in real-time and for use in predicting disinfection performance.

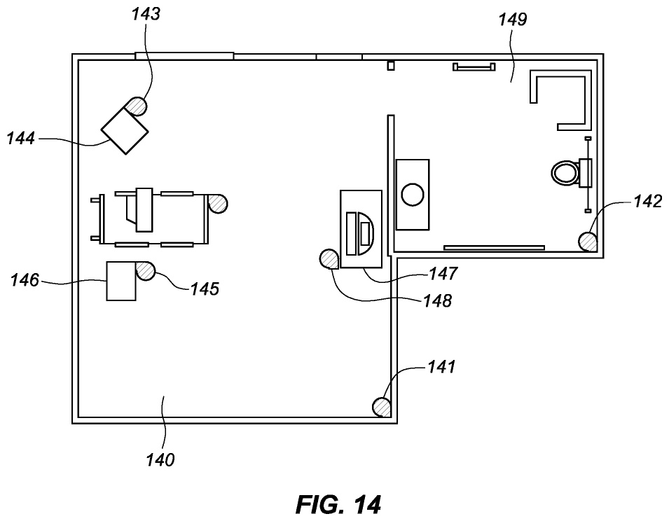

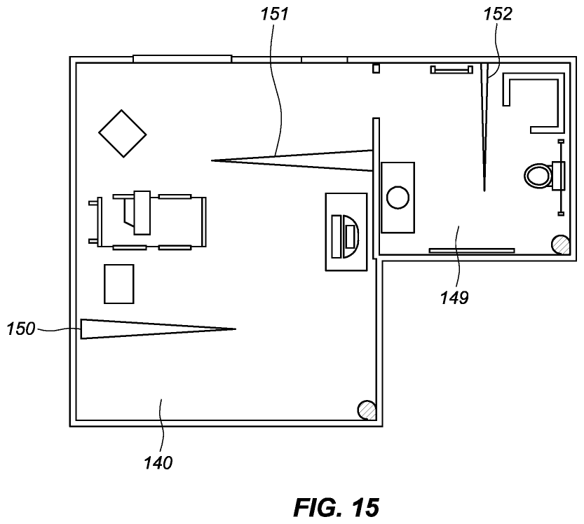

The data from the 3D spatial tracking array described above may be used to create a simulation of pathogen disinfection over the disinfected surface area. An exemplary floor plan is shown in FIG. 14 with optional UHF-Theta transducers 141 and 142 in the corners of rooms 140 and 149 respectively. In room 140, UHF-Theta transducer 143 is on chair 144. UHF-Theta transducer 148 is on table 147. UHF-Theta transducer 145 is attached to table 146. FIG. 15 shows the exemplary floor plan of FIG. 14. overlaid with a display of the simulated deficient disinfection areas 150, 151, and 152 which are all red in color, indicating 0.1 log pathogen kill.

For each pathogen, data exists which correlates UV fluence to log kill for the pathogen. This data may also be obtained through experimentation and refined by testing on different types of surfaces. The UV fluence applied to the surface may be computed from data including instantaneous speed along the surface, X-Y orientation of the disinfection device (azimuth), path overlap, and surface reflectivity. To compute the UV fluence, the UV lamp power is divided by the illumination area of the disinfection device and divided by the instantaneous speed at the point on the surface. If the direction of travel of the disinfection device is not perpendicular to the lamp axis, the angular orientation is introduced. The UV fluence is then summed at each point of the surface which is illuminated with the disinfection device located at that point on the spatial tracking array.

Surfaces that need to be wiped down prior to UV disinfection may use optional tracked sponge 90 or tracked mop 100, which can also record their paths and instantaneous velocities and surface contact to create a prerequisite liquid disinfectant simulation. The results may be displayed in combination with the UV disinfection simulation.

Computed disinfection is preferably displayed by displaying distinct colors representing a spectrum from excellent disinfection performance (6 log kill) to poor disinfection performance (0.1 log kill). These colors are preferably mapped onto highly localized areas such as pixels in an image which correspond to a rectangular grid of points, for example at 1 cm (0.4 inch) spacing, across the entire disinfected surface. The image is preferably displayed on a head-mounted display, on a screen on the disinfection device, or on a computer tablet display or other display. For example, the pathogen kill rate may be assumed to vary from 0.1 log (9%) to 6 logs (99.9999%) at each point on a surface. A distinct color may be assigned to each log kill rate for use in displaying the data. Exemplary log kill color assignments may be:

| Log Kill Rate | Color |
|---|---|
| 0.1 | Red |
| 1 | Orange |
| 2 | Yellow |
| 3 | Green |
| 4 | Blue |
| 5 | Indigo |
| 6 | Violet |

Alternately, another method is used.

During the disinfection simulation, the UV fluence that was emitted by the disinfection device is summed at each grid point over the area of a known surface, including the UV lamp intensity, disinfection device velocity at that point for each pass, disinfection device angle relative to the device path direction, and the area of effective UV illumination for the disinfection device 10. Optionally, a safety factor greater than 1.0 is used to multiply the required UV fluence at difficult-to-disinfect areas of the surface as demonstrated by swab calibration or other techniques. The effect of multiple passes is added as needed. The simulation results are preferably stored in a disinfection performance array. After all or a portion of the surface is simulated, the results are displayed to show areas of sufficient and insufficient disinfection. Many variants of this algorithm can work well. Many sensor combinations can be used. Data may be housed on the disinfection device or on a computer server, which may be local or remotely located. Data can be held in local memory on the disinfecting device or uploaded to local or remote computer.

Preferably, a model of the subject room or area is acquired prior to the use of the disinfection device 10. The geometry of the surface to be disinfected may be inputted into the computer by manual means such as 3D Computer Aided Design (3D CAD) systems, or by automatically scanning the space by 3D scanning with visual and infrared depth sensing cameras on the Microsoft HoloLens or spatial capture tools such as Canvas, or by another technique. Alternately, the space to be disinfected may be taken from existing floor plan CAD files. Alternately, it may be digitized with an RGB-D camera. Areas of special attention (or which require another type of device to clean) are preferably noted in the 3D CAD model such as crevices in chairs, toilets, sinks, showers, etc.

A mixed reality headset may be equipped with accelerometers, gyroscopes, magnetometers, and other sensors to register its position and orientation in a room. This information is then used to display the proper image which appears on the headset to overlay the corresponding surfaces of the room.

The spatial display of the disinfection effectiveness of existing whole room UV room robots can be performed by computing the UV fluence at each location in room and taking into account wall and ceiling reflections and UV absorbance of atmosphere in room along light path may be computed using a similar technique.

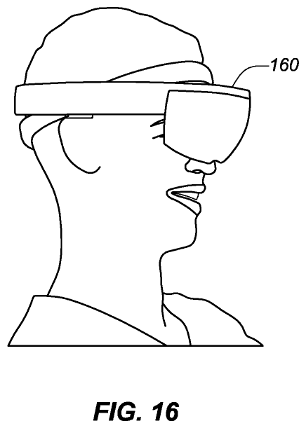

Preferably, the results from the disinfection simulation are immediately displayed to provide real-time feedback to the staff on the disinfection performance. The disinfection simulation display will either confirm proper disinfection or it will identify areas requiring further disinfection. In the preferred embodiment, a head-mounted mixed reality or augmented reality display 160 as shown in FIG. 16 is used to overlay the results of the disinfection simulation over the patient room or other facility.

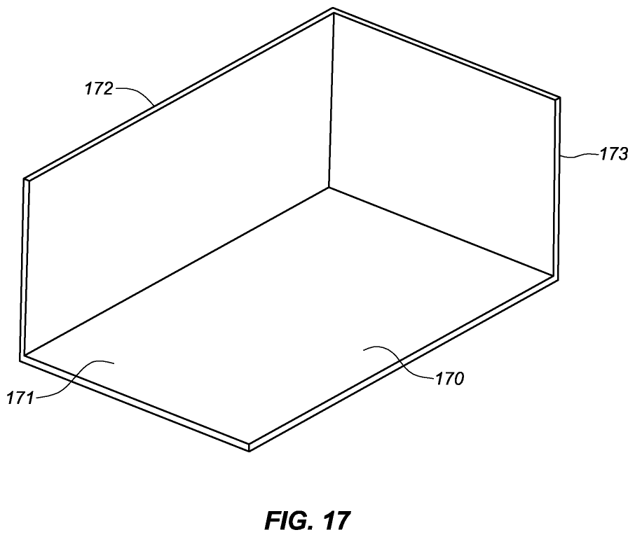

For tables and other objects requiring edge disinfection, the disinfection performance of the edges may be displayed on a planar tablet display by displaying a circumferential margin surrounding the disinfected main surface. FIG. 17 shows an exemplary visual display with mixed reality overlay of spatial disinfection data. In accordance with the color map described above, surface 172 is indigo in color, indicating 6 log pathogen kill. Areas 171 and 173 are red in color, indicating 0.1 log kill. Area 170 is yellow in color, indicating 2 log kill.

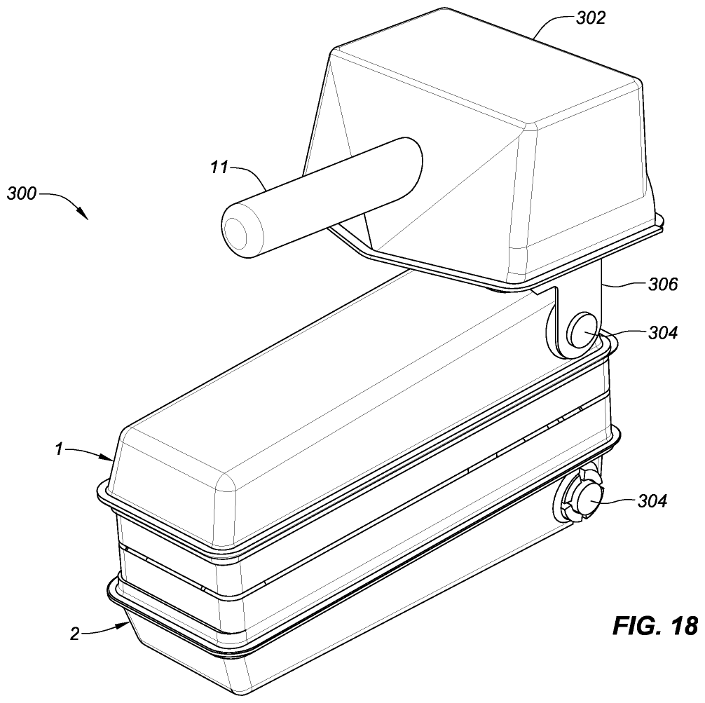

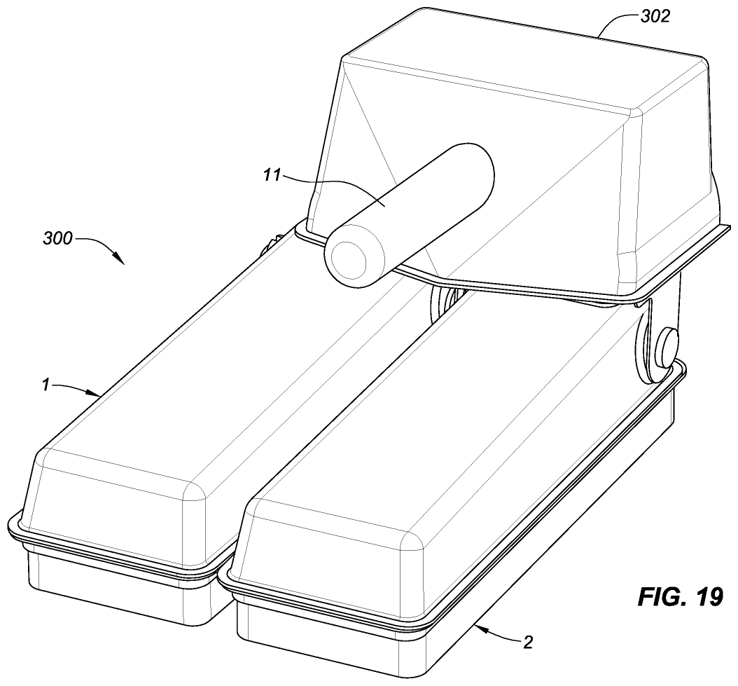

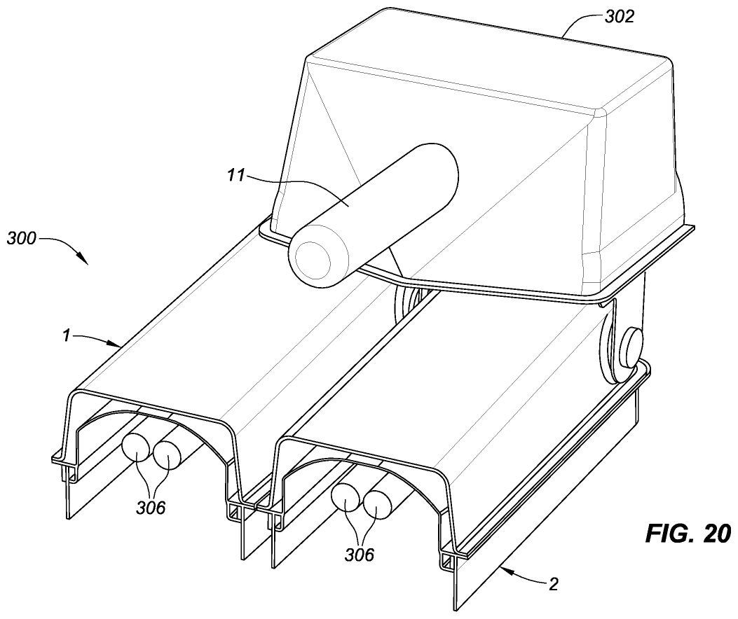

Another exemplary handheld UV disinfection device 300 is shown in FIGS. 18-20. Except as described below, the disinfection device 300 may be configured and utilized substantially as described about with regard to the disinfection device 10 of FIG. 1. A handle 11 may be affixed to or otherwise connected to a base 302. The disinfection device 300 may include two UV lamps 1, 2, where those two UV lamps 1, 2 are rotatable relative to one another. Each UV lamp 1, 2 may be rotatable relative to the base 302. An end of each UV lamp 1, 2 may be connected to a hinge 304 that is connected to the base 302 or that is held off from the base 302 by a bracket 306. The bracket 306 may be rotatably connected to the base 302 in a manner that allows for rotation of one or more of the UV lamps 1, 2, in a first direction about the corresponding hinge 304, and then in a second direction different from the first direction. Each UV lamp 1, 2 includes one or more UV lamp tubes 306, as described above with regard to the disinfection device 10 of FIG. 1. The hinge or hinges 304 allow the UV lamps to be moved apart from one another and spaced apart a small distance, to allow for easier and simpler disinfection of items such as bed rails, which may be placed between the spaced-apart UV lamps 1, 2. The hinge or hinges 304 also allow the disinfection device 300 to be folded to a stowed position for storage, such that the disinfection device 300 takes up less space, and such that the lamp tubes 306 face one another and are protected from accidental damage.

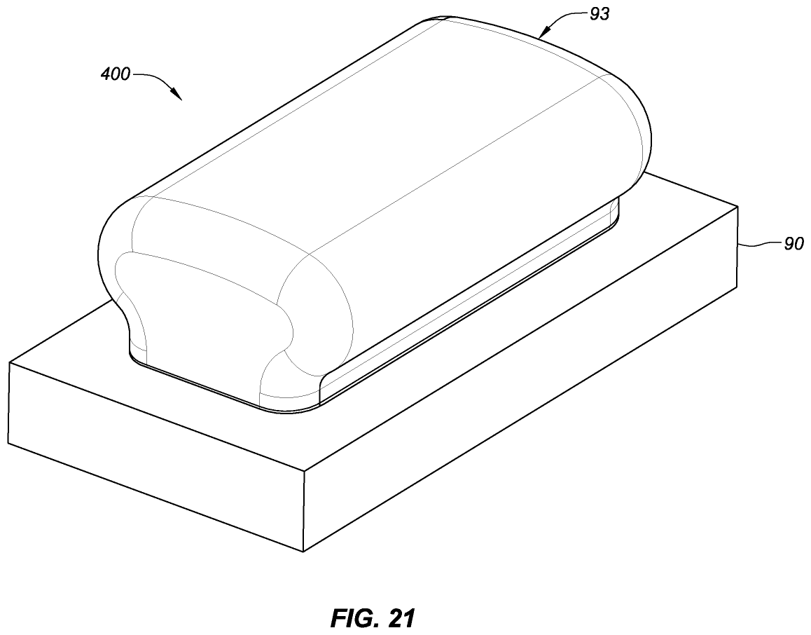

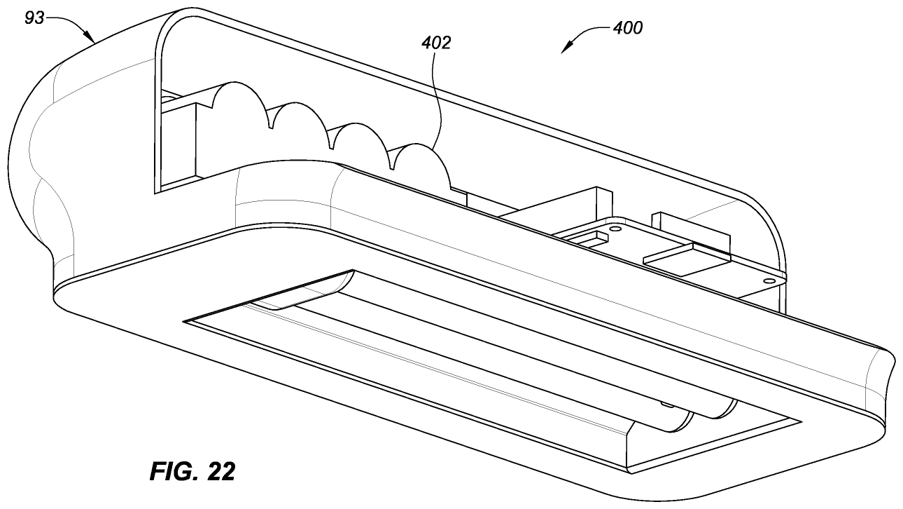

Referring also to FIG. 9, as set forth above, another exemplary handheld UV disinfection device 400 may include a sponge 90 attached to a housing 91. Referring also to FIGS. 21-22, the housing 91 may include one or more LEDs 402 or other sources of light. The LEDs 402 may be configured to emit two or more different colors; alternately, a plurality of LEDs 402 are provided, each configured to emit a different color of light. The LEDs 402 may be used in lieu of a display 14 or a mixed reality display as described above, or in conjunction with such displays. As described above with regard to a mixed reality display, different displayed colors indicate spatial disinfection data by color. The LEDs 402 are configured to emit light into the housing 91 and/or sponge 90, such that the user can view the housing 91 and/or sponge 90 directly and see colors associated with the disinfection simulation. For example, a red LED 402 may emit red light when the disinfection simulation shows a less than optimal amount of disinfection, so that the user can continue to utilize the disinfection device 400 and disinfect the surface further. A green or indigo LED 402 may emit light when the disinfection simulation shows that the optimal amount of disinfection has been achieved

As used in this document, and as customarily used in the art, the word “substantially” and similar terms of approximation refer to normal variations in the dimensions and other properties of finished goods that result from manufacturing tolerances and other manufacturing imprecisions, and that result from normal variations in the measurement of such dimensions and other properties of finished goods.

While the invention has been described in detail, it will be apparent to one skilled in the art that various changes and modifications can be made and equivalents employed, without departing from the present invention. It is to be understood that the invention is not limited to the details of construction, the arrangements of components, and/or the method set forth in the above description or illustrated in the drawings. Statements in the abstract of this document, and any summary statements in this document, are merely exemplary; they are not, and cannot be interpreted as, limiting the scope of the claims. Further, the figures are merely exemplary and not limiting. Topical headings and subheadings are for the convenience of the reader only. They should not and cannot be construed to have any substantive significance, meaning or interpretation, and should not and cannot be deemed to indicate that all of the information relating to any particular topic is to be found under or limited to any particular heading or subheading. Therefore, the invention is not to be restricted or limited except in accordance with the claims.

What is claimed is:

1. A disinfection device, comprising:
a housing;
at least one reflector within said housing;
at least one ultraviolet emitter mounted in each said reflector; and
at least one position sensor associated with said housing.

2. The disinfection device of claim 1, wherein at least one said ultraviolet emitter is a lamp.

3. The disinfection device of claim 1, wherein at least one said ultraviolet emitter is an LED.

4. The disinfection device of claim 1, wherein said window is fabricated from at least one of the materials selected from the group consisting of fused quartz, FEP film, and colorless polyimide.

5. The disinfection device of claim 1, further comprising a disinfectant applicator associated with said housing.

6. The disinfection device of claim 5, wherein said disinfectant dispenser is positioned laterally between said ultraviolet emitters.

7. The disinfection device of claim 5, wherein said disinfectant dispenser is passive.

8. The disinfection device of claim 5, wherein said disinfectant dispenser is active.

9. The disinfection device of claim 1, wherein at least one position sensor is a two-axis position sensor.

10. The disinfection device of claim 1, wherein at least one position sensor is a three-axis position sensor.

11. The disinfection device of claim 1, wherein at least one position sensor is an optical sensor.

12. The disinfection device of claim 1, wherein at least one position sensor is a 3D accelerometer.

13. The disinfection device of claim 1, wherein at least one position sensor is a 3D gyroscope.

14. The disinfection device of claim 1, wherein at least one position sensor is an ultra-wideband transponder.

15. The disinfection device of claim 1, further comprising a window that is substantially transparent to ultraviolet light, adjacent to each said reflector, through which ultraviolet light emitted by each said ultraviolet emitter travels

16. A disinfection device, comprising:
a housing;
a disinfectant applicator associated with said housing; and
at least one position sensor associated with said housing.

17. The disinfection device of claim 16, wherein said at least one position sensor causes position information to be communicated to a user.

18. The disinfection device of claim 16, wherein said at least one position sensor causes position information to be communicated to a user.

19. The disinfection device of claim 16, wherein said at least one position sensor causes position information to a user.

20. The disinfection device of claim 19, further comprising at least one LED associated with said housing, wherein said at least one position sensor communicates position information to said at least one LED, which displays position information to a user.
